(19) United States
(12) Patent Application Publication
(10) Pub. No.: US 2011/0290255 A1
(43) Pub. Date: Dec. 1, 2011

(54) TRACHEAL CANNULA

(76) Inventor: Eric Bezicot, Anthony (FR)

(21) Appl. No.: 13/202,588

(22) PCT Filed: Feb. 5, 2010
(86) PCT No.: PCT/FR10/00084

§ 371 Date: Aug. 22, 2011

(30) Foreign Application Priority Data
Feb. 20, 2009 (FR) 09/00779

(51) Int. Cl. A61M 16/04

(52) U.S. Cl. 128/207.14

(57) ABSTRACT

The present invention relates to a cannula (1) including a tubular body (2) with a first end (3) intended for being inserted in a surgical opening or natural orifice of a patient and a second end (4) intended for remaining on the outside and engaging with the opening by means of a flange, characterised in that the flange has at least one gripping tab (7).

### TECHNICAL FIELD

The present invention relates to a tracheal cannula.

### BACKGROUND

A tracheal cannula is a tube allowing air to flow through an orifice made in an anterior face of the trachea after tracheotomy or laryngectomy.

Thus, a tracheal cannula aims at allowing breathing after having been introduced into the trachea, when there exists an obstacle in the larynx.

Tracheotomy may be temporary or definitive.

This cannula may be made in various materials and may for example be in plastic material, metal, silicone, rubber, PVC.

The cannula may be rigid or flexible, straight or curved, with the variable length and caliber depending on the patient and on the use.

A tracheotomy cannula may be definitive, and it will then be preferentially made from silver, or used occasionally and may then be made in rubber, silicone.

While a silver cannula may be used for several years, a cannula made in other materials may be led to having to be removed temporarily with view to its replacement and to its maintenance, or definitely when the tracheotomy is temporary.

A first cannula replacement generally occurs about two days after the operation. Cannula care services requiring the removal of the cannula are necessary daily during the days following the operation.

It is therefore understood that removal of the cannula may be frequent and there exists a need for facilitating its extraction and its putting back into place.

More particularly, in the case of complete care and when the cannula has to be removed for a rather long time, there exists a risk of reducing or even closing the tracheostoma again. Therefore, there exists a need for a device with which it is possible to reduce the intervention time during which the cannula is removed.

Moreover, it will be noted that as the cannula has to be introduced into the trachea, its handling is generally difficult and it only has few gripping points.

Generally, for the comfort of the patient, removing the cannula and putting it back into place should be able to be carried out as rapidly as possible.

### BRIEF SUMMARY

The present invention aims at giving the possibility of reducing the time for removing a tracheal cannula and putting it back into place and to do this, it comprises a cannula comprising a tubular body having a first end intended to be introduced inside a surgical or natural orifice of a patient and a second end intended to remain on the outside and come into contact with the orifice via an edge, characterized in that this edge has at least one gripping tab.

Thus, by fitting out the cannula with a gripping tab, the handling of the cannula during its removal and its putting back into place is considerably facilitated, which results in greater intervention rapidity, appreciable for the patient.

Advantageously, the tab is oriented towards the inside of the edge. It was actually noticed that the presence of a tab did not notably perturb the flow of air through the cannula. Moreover, with this, it is possible to keep the external peripheral edge and to fit a filter or an artificial nose thereon, for example.

Preferentially, the tab has a rounded shape.

Of course, various forms of tabs may be contemplated. Sention may be made of tabs of a substantially rectangular, triangular shape with a rounded tip, etc.

Advantageously, the tab is made in a flexible material.

Still advantageously, the tab is made in one single piece with the cannula, for example by molding.

According to a first alternative embodiment, the cannula is made from silicone.

According to a second alternative embodiment, the cannula is made from a plastic material, notably of the PVC type.

Advantageously, the second end has a funnel shape.

Preferentially, the cannula is a tracheal cannula.

### BRIEF DESCRIPTION OF THE DRAWINGS

The present invention will be better understood by means of the detailed description which is discussed below, with reference to the appended drawing wherein:

FIG. 1 is an illustration of a tracheal cannula according to the invention,

FIG. 2 is a schematic longitudinal sectional illustration of the tracheal cannula of FIG. 1,

FIG. 3 is a front illustration of the tracheal cannula of FIG. 1,

FIG. 4 is an enlarged partial illustration of the second end of the tracheal cannula at the gripping tab.

### DETAILED DESCRIPTION

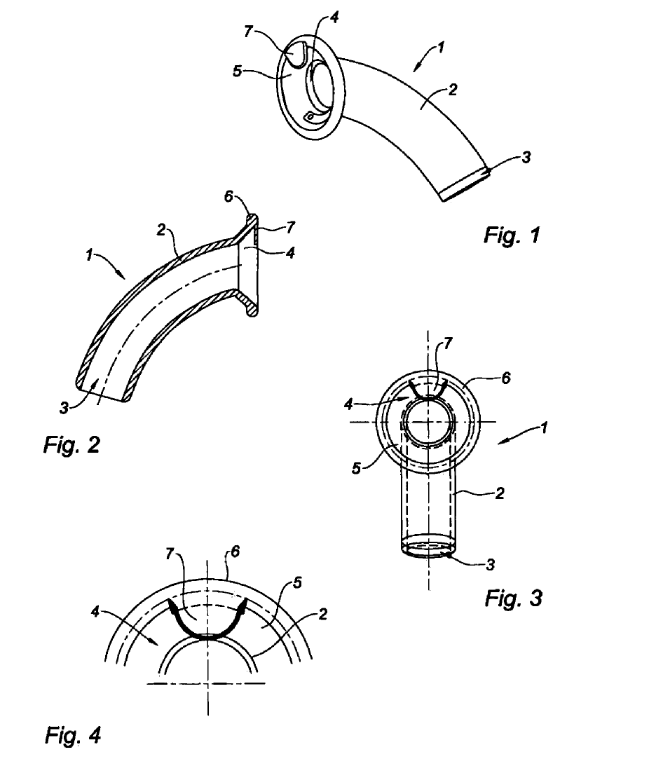

A tracheal cannula 1 as illustrated in FIGS. 1 to 3, comprises a slightly curved tubular body 2 made in medical grade silicone.

The cannula 1 has a first end 3 intended to be inserted through a tracheostoma, and a second end 4 intended to remain on the outside and come into contact with said tracheostoma.

The end 4 has a funnel-shaped head 5 having a peripheral bulge 6 being used as a support, and generally capable of allowing the attachment of an artificial nose, a filter or other device.

According to the invention, the bulge 6 is fitted out with a gripping tab 7 oriented towards the inside of said peripheral bulge 6, and having substantially the shape of a half circle.

The tab 6 will preferentially be made integral with the cannula by thermomolding of the silicone.

Thus, a user who wishes to remove the cannula 1 will grasp the tab 7 between his/her fingers in order to exert traction thereon in the direction of the removal.

Conversely, when the cannula 1 is put back into place, the tab will facilitate the final operation for putting it back into place.

Although the invention has been described with a particular exemplary embodiment, it is quite obvious that it is by no means limited to it and that it comprises all the technical equivalents of the described means as well as their combinations, if the latter enter the scope of the invention. It is notably possible to provide at a neck of the cannula, for example, apertures for letting through a chord.

1. A cannula comprising a tubular body having a first end intended to be introduced into an inside of a surgical or natural orifice of a patient and a second end intended to remain on the an outside and to come into contact with the orifice via an edge, wherein the edge has at least one gripping tab.

2. The cannula according to claim 1, wherein the tab is oriented towards an inside of the edge.

3. The cannula according to claim 1, wherein the tab has a rounded shape.

4. The cannula according to claim 1, wherein the tab is made in a flexible material.

5. The cannula according to claim 1, wherein the tab is made in one single piece with the cannula by molding.

6. The cannula according to claim 1, wherein the cannula is made from silicone.

7. The cannula according to claim 1, wherein the cannula is made from a plastic material of the PVC type.

8. The cannula according to claim 1, wherein the second end has a funnel shape.

9. The cannula according to claim 1, wherein the cannula is a tracheal cannula.
